(19) United States
(12) Patent Application Publication
(10) Pub. No.: US 2015/0077756 A1
(43) Pub. Date: Mar. 19, 2015

(54) SYSTEM AND METHOD FOR CONTINUOUS REAL-TIME MONITORING OF WATER AT CONTAMINATED SITES

(71) Applicant: LUMENSE, INC., Atlanta, GA (US)

(72) Inventors: Daniel P. Campbell, Atlanta, GA (US); Jayme Caspall, Decatur, GA (US); Janet Cobb-Sullivan, Marietta, GA (US); Kenneth E. Johnson, Kennesaw, GA (US); Robert E. Johnson, Atlanta, GA (US); Larry Starr, Colorado Springs, CO (US); Michael Slawson, Atlanta, GA (US); Ruoya Wang, Marietta, GA (US)

(21) Appl. No.: 14/303,677

(22) Filed: Jun. 13, 2014

Related U.S. Application Data
(60) Provisional application No. 61/834,451, filed on Jun. 13, 2013.

(51) Int. Cl. G01N 33/18; G01B 9/02

(52) U.S. Cl. CPC G01N 33/1826; G01B 9/02
USPC 356/450

(57) ABSTRACT

A system and method for the monitoring of contaminants in water. The water monitoring system includes a contaminant sensor that is configured to detect trace amounts of contaminant in the water that is pumped through it in real time. The real time contaminant sensor includes an interferometer configured to track the amount of contaminants that is pumped into the real time contaminant sensor.

### CLAIM OF PRIORITY

This application claims priority from U.S. Provisional Application No. 61/751,430, filed Jun. 13, 2013, which is relied upon and incorporated herein in its entirety by reference.

### BACKGROUND

Freshwater sources, both underground (groundwater) and at the surface, can, unfortunately, become contaminated, by both point and non-point source pollutants, almost anywhere, but are quite often contaminated at active energy producing and testing facilities throughout the world. The United States of America's Department of Energy (DOE), for example, has the challenge of containing and remediating contaminated groundwater and surface water at over 3,650 active sites. These sites are dispersed over eleven states and have different geologies, contamination characteristics, and stages of treatment, all which are in need of constant monitoring of the water resources for contaminants.

The contaminated water resources can include a wide range of contaminants including heavy metals, radioactive materials, and a variety of organics. Many contaminated water resources (e.g., DOE's Savannah River Site), however, have Volatile Organic Compound (VOC) contaminants, specifically from the chlorinated ethylene family. Trichloroethylene (TCE) is often the most common groundwater contaminant. TCE and perchloroethylene (PCE) can form Dense Non-Aqueous Phase Liquid (DNAPL) pools deep in the ground that are difficult and expensive to treat and fully remove. DNAPLs present risk to humans and are reported to cause neurological disorders, immune system disorders, birth defects, liver toxicity, and cancer. Both TCE and PCE eventually degenerate into breakdown products (cis-dichlorethylene (DCE), trans-DCE and vinyl chloride) that also cause health concerns.

The Maximum Contaminant Level established by Environmental Protection Agency (EPA) for TCE is five parts per billion (ppb), yet some contaminated sites have reported levels of TCE exceeding one part per million (ppm). The TCE problem is significant, as anywhere from nine to thirty-four percent of the US drinking water supply is reported to be contaminated by TCE. Such findings show that it is necessary to continue the careful and effective surveillance of TCE and other toxic compounds at contaminated and uncontaminated water recourses.

However, the continuous monitoring of such water resources is very costly. Sampling methodology and frequency and laboratory expenditures compose the main elements of monitoring costs. For example, current DOE methodology has trained personnel traveling to each contaminated site, “purging” each well to be analyzed, and taking a sample from that well or surface water location. Each sample is labeled, tracked, and transported to a laboratory for analysis. Results are typically available in a week. Sampling cost is especially affected by the method used to purge the well prior to collecting the sample. Costs vary widely, from $349 to $8,760 per well per sample, according to one analysis. In an extreme case, the cost of purging a single well was reported to have exceeded $8,000. In 2004, the Savannah River Site reported that its costs ranged between $100 and $1,000 per sample across approximately 4,000 sampling locations.

Sampling frequency is another factor significantly impacting the cost of groundwater monitoring. Frequencies vary from every fourteen days to every four years, depending on the treatment stage and the regulatory requirements set for specific health threats. Labor and “truck roll” costs are typically $500 per sample. Laboratory costs are typically a few hundred dollars or higher per sample.

Furthermore, approximately ten percent of DOE sites are estimated to be located in complex geologies, with DNAPL pools that are extremely difficult to precisely locate and remove. As a result, these sites must be monitored for years after the end of treatment, even though chemical or biological decomposition methods may have been used for several years. Typically, monitoring wells are placed throughout a complex site to allow for tracking of the plume. In summary, traditional approaches force DOE and other entities responsible for remediation efforts into an expensive, “one size fits all” regimen, requiring incremental costs for each measurement.

Therefore, there is a need for a field-ready monitoring technology that can make accurate, automated, portable, and near-real-time measurements, thus dramatically reducing cost while enabling monitoring entities to better assess and reduce environmental risk.

### SUMMARY OF THE INVENTION

The present invention provides a ground and surface water monitoring system. The ground and surface water monitoring system includes a sensor that is configured to detect the entry of contaminants, which can include, but are not limited to, aromatic hydrocarbons (benzene, toluene, ethylbenzene, and xylene) and chlorinated ethylenes (TCE, PCE, cis-DCE, trans-DCE, and vinyl chloride), into a water supply. In an aspect, the sensor is configured to detect a change in the total contaminant level. In another aspect, the sensor is configured to detect the composition of a contaminant mixture, as well as the change of the composition of the contaminant mixture.

In an aspect, the sensor is configured to provide a concentration level of a known contaminant, or single unknown contaminant, found in a water source. In another aspect, the sensor is configured to identify and quantify compounds in a random mixture of contaminants.

In an aspect, the ground and surface water monitoring system is configured to be fully automated. In an aspect, the ground and surface water monitoring system is configured to continually monitor the water resources. In another aspect, the ground and surface water monitoring system is ruggedly configured to work reliably and robustly in the field.

These and other objects and advantages of the invention will become apparent from the following detailed description of the preferred embodiment of the invention.

Both the foregoing general description and the following detailed description are exemplary and explanatory only and are intended to provide further explanation of the invention as claimed. The accompanying drawings are included to provide a further understanding of the invention and are incorporated in and constitute part of this specification, illustrate several embodiments of the invention, and together with the description serve to explain the principles of the invention.

### BRIEF DESCRIPTION OF THE DRAWINGS

FIG. 1 is a schematic view of a ground and surface water monitoring system utilizing a real-time sensor according to an aspect.

FIG. 2 is a front plan view of the real-time sensor of FIG. 1.

FIG. 3 is a schematic view of the subsystems of ground and surface water monitoring system of FIG. 1.

FIG. 4 is a flow diagram of the water circulation system of the ground and surface water monitoring system of FIG. 3.

FIG. 5 is a cross-sectional side schematic view of the chemical sensor module of the ground and surface water monitoring system according to one embodiment of the present invention.

FIG. 6 is a schematic top view of the waveguide of FIG. 5.

FIG. 7 is a top view of a waveguide with four separate channel pairs.

FIG. 8 is a schematic representation of a single mode waveguide with buried evanescent field according to an aspect.

FIG. 9 is a flow chart of a method performed by components of an ammonia monitoring system according to an aspect.

FIGS. 10-12 are representations of the display of the real-time contaminant sensor of FIG. 1.

FIG. 13 is a flow diagram of a method performed by the ground and surface water monitoring system of FIG. 2.

FIG. 14 is a flow diagram of a method performed by the ground and surface water monitoring system of FIG. 2.

FIG. 15 is a block diagram of a computing device according to an embodiment of the present invention.

FIG. 16 illustrates the results of testing of an example of the contaminant sensor according to an aspect.

### DETAILED DESCRIPTION OF THE INVENTION

Before the present methods and systems are disclosed and described, it is to be understood that the methods and systems are not limited to specific synthetic methods, specific components, or to particular compositions. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal embodiment. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

As will be appreciated by one skilled in the art, the methods and systems may take the form of an entirely hardware embodiment, an entirely software embodiment, or an embodiment combining software and hardware aspects. Furthermore, the methods and systems may take the form of a computer program product on a computer-readable storage medium having computer-readable program instructions (e.g., computer software) embodied in the storage medium. Any suitable computer-readable storage medium may be utilized including hard disks, CD-ROMs, optical storage devices, or magnetic storage devices.

Embodiments of the methods and systems are described below with reference to block diagrams and flowchart illustrations of methods, systems, apparatuses and computer program products. It will be understood that each block of the block diagrams and flowchart illustrations, and combinations of blocks in the block diagrams and flowchart illustrations, respectively, can be implemented by computer program instructions. These computer program instructions may be loaded onto a general purpose computer, special purpose computer, or other programmable data processing apparatus to produce a machine, such that the instructions which execute on the computer or other programmable data processing apparatus create a means for implementing the functions specified in the flowchart block or blocks.

These computer program instructions may also be stored in a computer readable memory that can direct a computer or other programmable data processing apparatus to function in a particular manner, such that the instructions stored in the computer-readable memory produce an article of manufacture including computer readable instructions for implementing the function specified in the flowchart block or blocks. The computer program instructions may also be loaded onto a computer or other programmable data processing apparatus to cause a series of operational steps to be performed on the computer or other programmable apparatus to produce a computer-implemented process such that the instructions that execute on the computer or other programmable apparatus provide steps for implementing the functions specified in the flowchart block or blocks.

Accordingly, blocks of the block diagrams and flowchart illustrations support combinations of means for performing the specified functions, combinations of steps for performing the specified functions, and program instruction means for performing the specified functions. It will also be understood that each block of the block diagrams and flowchart illustrations, and combinations of blocks in the block diagrams and flowchart illustrations, can be implemented by special purpose hardware-based computer systems that perform the specified functions or steps, or combinations of special purpose hardware and computer instructions.

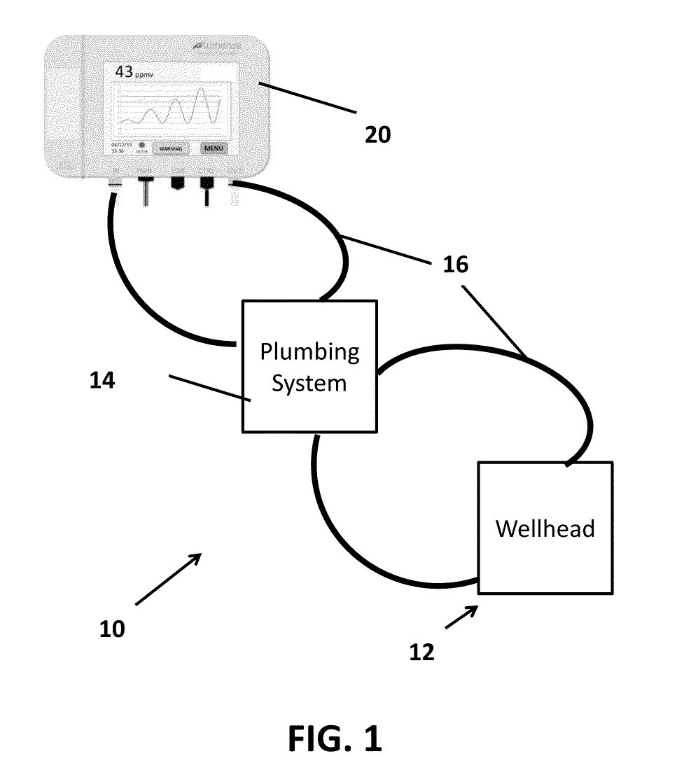

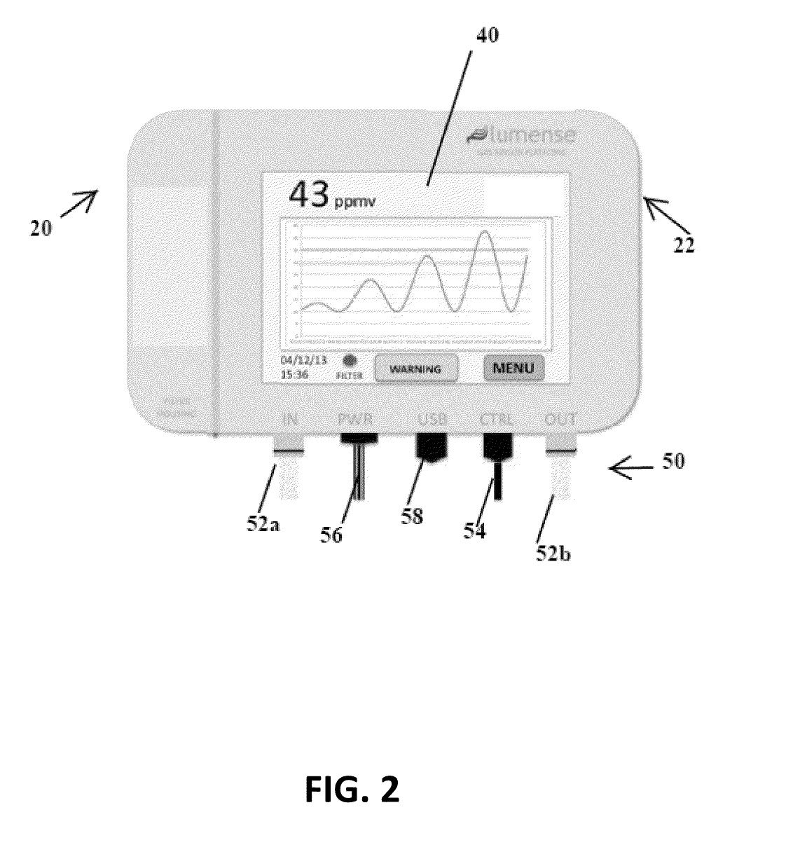

Referring to FIGS. 1 and 2, the present invention is directed to a ground and surface water monitoring system 10. While FIG. 1 shows the ground and surface water monitoring system 10 configured for monitoring water at wellheads 12 at known active contaminant sites, the ground and surface water monitoring system 10 can be used in a variety of settings in which the continuous monitoring for contaminants in a fluid (i.e., gas or liquid) is needed. For example, the ground and surface water monitoring system 10 can be configured for use in water treatment plants, public water sources, oil and gas drilling operations, residential water supplies, or water sources used for healthcare, industrial, or agricultural applications. Further, the ground and surface water monitoring system 10 can be utilized in utility industry settings. Such utility industry settings include, but are not limited to, power generation facilities.

In an aspect, the ground and surface water monitoring system 10 is configured to be capable of being located near a monitoring wellhead 12, and of receiving a continuous flow of water from that wellhead 12. In an aspect, the ground and surface water monitoring system 10 includes a plumbing system 14 to deliver the monitored water to the real-time sensor 20. In an aspect, the ground and surface water monitoring system 10 is further configured to be employed at remote, unattended locations. In such aspects, the ground and surface water monitoring system 10 can be configured for wireless communications. For example, the wireless communications can include, but are not limited to, cellular communication means (CDMA, GPRS, LTE, etc.), Bluetooth, Wi-Fi, satellite, and other types of wireless connectivity. In aspects in which the ground and surface water monitoring system 10 is not needed to be used in remote locations (e.g., industrial settings), wired connectivity means (LAN, T1, intranet, etc.) can also be used.

In an aspect, the ground and surface water monitoring system 10 is configured for use at a wellhead/water source 12. In such an aspect, the ground and surface water monitoring system 10 can include a plumbing system 14 to deliver the monitored water to the sensor 20. The plumbing system 14 can include tubing 16 that reaches the water source 12, and may be selected depending on the types of contamination believed to be present. Such tubing 16 can include, but is not limited to, PVC, Tygon, PTFE, polyethylene, and the like. In an aspect, the tubing 16 can be of a flexible nature in order to avoid any potential obstructions between the sensor 20 and the water source 12. Various fastening means, including, but not limited to, mounting brackets, may be used to secure the components of the plumbing system 14, including the tubing 16, in place at the monitoring site. While the sensor 20 can be mounted at various locations at the wellhead/waters source 12, it is preferable that it is placed at a position of easy access for routine maintenance, if needed. The ground and surface water monitoring system 10 is not limited to the just described configuration.

In an aspect, as shown in FIG. 2, the sensor 20 includes a housing 30, a user interface 40, and external connections 50. The housing contains various hardware and software components discussed in further detail below. Since the ground and surface water monitoring system 10 is configured for use of the monitoring of wellheads 12, which can be found in harsh environmental conditions (exposed, remote locations), the housing 30 can be made of a rugged and rigid material. The sturdy material can include, but is not limited to, sheet metal, aluminum, or molded plastic, that is sufficient to protect the inner components of the sensor 20 and meet safety and regulatory requirements. In an aspect, the housing 30 can include a removable cover, giving access to certain components of the sensor 20, including a filter. In the preferred embodiment of the present invention, the housing 30 is configured to notify (e.g., via a switch) the various hardware and software components of the sensor when the cover is removed or reinstalled.

As discussed above, in an aspect, the contaminant sensor 20 can include a user interface 40. In such an aspect, the user interface 40 comprises a single, large, backlit color touchscreen on which all user controls and displays reside. While the preferred embodiment of the present invention uses an interactive touchscreen 40, the sensor 20 can include other types of user interfaces 40, including, but not limited to, a combination keypad and display screen, and the like. In some embodiments of the present invention, it may be desirable for the sensor 20 not to have any direct human-accessible interface, limiting the control of the sensor 20 to authorized individuals remotely through a wireless or wired connection, discussed in more detail below. Specific controls and displays, and their functions, are discussed in more detail below. While the dimensions of the user interface 40 and the overall housing 30 can be of various combinations, in one embodiment the touchscreen 40 is approximately 6″×4.5″ and the housing 30 is 7″×9″ in area, and 3″ deep.

As shown in FIG. 2, the sensor 20 provides external connections 50 that facilitate the monitoring of contaminant(s) within the water source being monitored. As shown in FIG. 2, the bottom of the housing 30 provides five external connections 50, including two fluidic connections 52a, 52b, a controller connection 54, a power connection 56, and an auxiliary data port 58. However, other embodiments of the sensor may include more or fewer external connections, as well as connections of different types.

The fluidic connections include an intake (“IN”) port 52a and an exhaust (“OUT”) port 52b, which connect to components of the plumbing system 14, which include the filter, valves, the flowcell which houses the waveguide, and, possibly, a pump, discussed in more detail below. In an aspect, the fluidic connections 52a, 52b can connect to an intake hose and an exhaust hose, respectively, that are connected to the plumbing system 14. These ports 52a, 52b may include nipples that connect to the respective hoses 16. In an aspect, it is preferable that the nipples and hoses are of different sizes to preclude cross connection. Further, it is preferable that the hoses 16 and all other parts exposed to the water on the inlet side be made of materials that do not leach off or absorb contaminants. This material, as discussed above, can include, but is not limited to, PTFE (polytetrafluoroethylene), polyethylene, or Tygon tubing.

The communication connector 54, labeled “CTRL” in FIG. 2, outputs real-time contaminant information, including level, concentration, and contaminant makeup. The communication connection 54 can be achieved wirelessly by adding an external wireless dongle at the CTRL port, or by using an internal modem. Further, in some aspects, the sensor 20 will not have an exposed communication connector 54 in order to prevent any unauthorized electronic access to the sensor 20.

According to an aspect, the power connector 56 is configured to accept a plug-in power cable. The power cable can be a standard 110 VAC, 3-prong cable approved for use in the United States. Other power cables suitable for other countries may be utilized as well. In some embodiments of the present invention, the power cable is hardwired into the sensor 20, with the appropriate strain relief. Power can be supplied to the sensor 20 from an on-site source, or from an electrical grid. In another aspect, the power connector 56 can be configured to be connected to a removable power source, such as a battery or an external adapter. In another aspect, the sensor 20 can have an internal power source. The internal power source can include, but is not limited to, a rechargeable battery, a replaceable battery, or some other means. For example, in the case of a rechargeable battery, a solar panel assembly can be used in connection with the rechargeable battery.

In an aspect, the sensor 20 can include auxiliary data ports 58, which enable additional access to sensor data and software. The auxiliary ports 58 can be configured to be compatible with standard electrical interfaces, including USB, Ethernet, Firewire, and the like. Some embodiments of the sensor 20 can include a dedicated memory stick configured to be removably coupled to the auxiliary data port. Such a memory stick can be used in a backup system to retain collected information. For example, in instances when the communication means of the sensor 20 becomes unavailable or broken, the data can still be collected and available by using a memory stick. The memory stick can be configured to connect to the contaminant sensor 20 to download data.

The auxiliary data port 58 is also configured to provide the interface for calibration and diagnostics, as well as the uploading of new software. The auxiliary data port 58 can be provided as a separate output from the communications connector 52 to allow a user to connect to the sensor 20 and download historical or real-time data without interrupting the output signal to the communications connector 52.

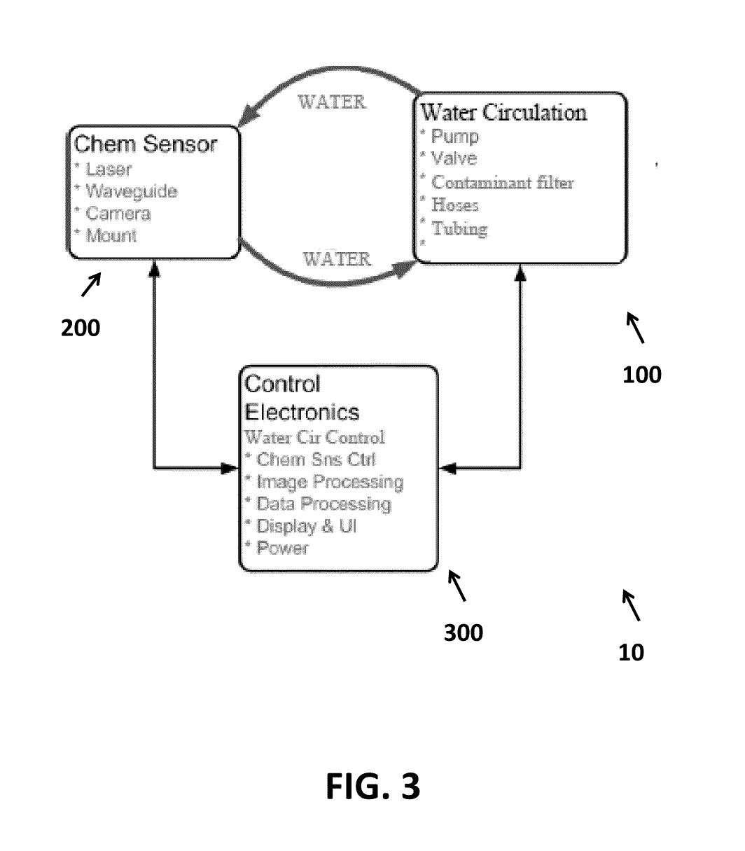

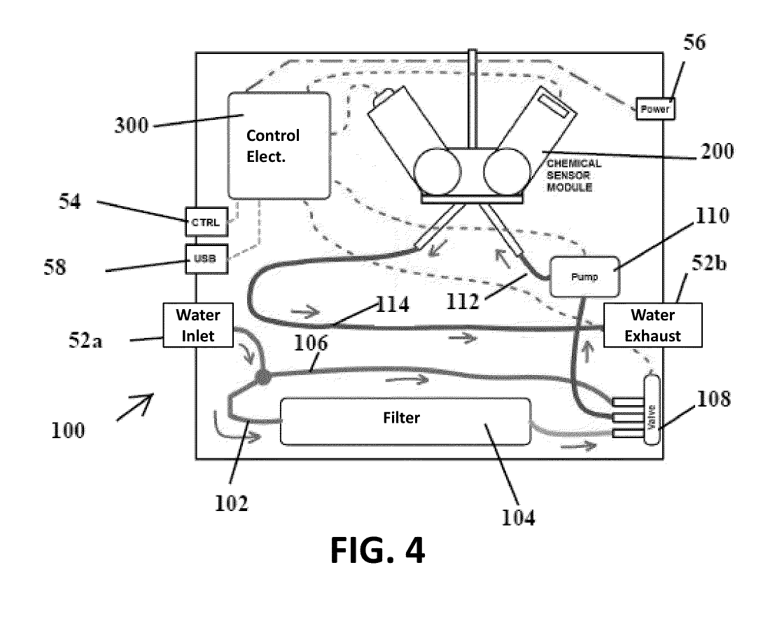

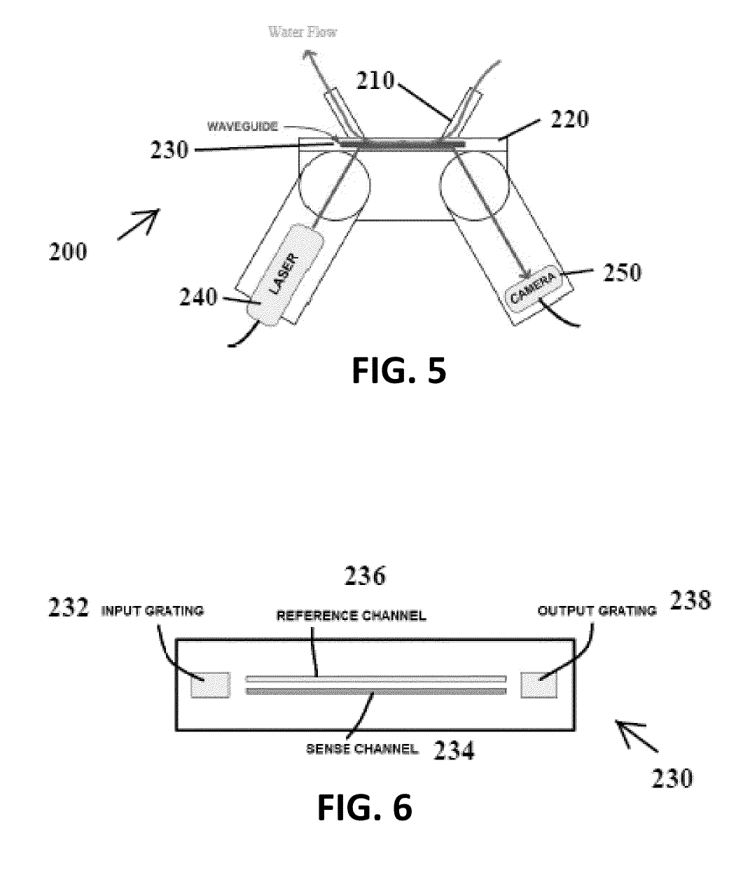

As discussed above, the sensor 20 includes hardware and software components. In an aspect, the hardware components of the sensor 20 can include three interdependent subsystems organized by hardware function—not by physical location or implementation. As shown in FIG. 3, the subsystems are an Water Circulation Subsystem (WCS) 100, which includes a pump (optional) and valve that interact with the all external and internal hoses (shown in FIG. 4), a chemical sensor module 200 (CSM), which includes a laser, waveguide, camera, and mount (shown in FIGS. 5-6) and Electronics Controller 300 (EC), an embedded system or single board computer which controls the WCS 100, the CSM 200, image processing from the CSM 200, data processing from all sensors, the touchscreen display 40, user Interface connections 50, and power conditioning. The EC 300 provides power and commands to all electronic components of the WCS 100, and receives data from the CSM 200. The EC 300 also outputs data to the communication 54 and auxiliary data connectors 58, and receives commands from the auxiliary data connector 58. In an embodiment, 110 VAC Power enters through the power connector 56 and is converted to required levels by internal electronics.

FIG. 4 is a block diagram showing connections and flow in the WCS 100. In one embodiment, water enters on the left through the water intake port 52a. In an aspect, the water intake port 52a can include a particle filter (not shown). The particle filter can be configured to prevent sediment and other solid matter from clogging the sensor. Once water has entered the water intake port 52a, and gone through the particle filter, the water is immediately split into two paths: one path 102 going through a filter 104, the other path 106 bypassing the filter 104. Both paths 102, 106 terminate in inlets to a valve 108, which selects either the filtered water (i.e., the water that passes through the filter 104) or the unfiltered water to pass through to the waveguide, which is contained in a flow cell, both of which are contained in the CSM 200, discussed in more detail below. The filter 104 takes the contaminants out of the water stream of the first path 102, but should not affect temperature or other characteristics. The filter material may be comprised of, but is not limited to, charcoal or activated carbon.

By having a filtered path 102 and a non-filtered path 106, the sensor 20, and more specifically the CSM 200, can self-calibrate on a regular basis to reduce sensor drift and maintain accuracy. For each sense cycle, the filtered water passed through the filtered path 102/104 is first measured to establish a zero-contaminant baseline, in order to cancel waveguide drift. The filtration system may also adjust the pH levels of the water being monitored, as well as the temperature. The filtered water path 102/104 and the unfiltered water path 106 should be balanced in terms of the pressure drop through the path, temperature, pH levels, and the travel time for water through the respective path 102, 106. A pump 110, which can be optional if the system 10 is gravity fed, and whose location in the stream can change (i.e., the pump 110 can be external to the sensor 20 and a part of an external plumbing system 14, or the pump 110 can be a separate component within the sensor 20), either pushes or pulls the water from the water source through the sensor 20, including to the CSM 200, for contaminant level measurement and composition. The water exits the CSM 200 and passes through the Water Exhaust Port 52b. In an alternative embodiment, the sensor 20 contains two valves, and water is continually pumped through both paths so that there is no latency between the environmental characteristics of the water in each path. Water from the path not being pulled or pushed through the CSM 200 bypasses the CSM 200 and is coupled with water coming out of the CSM 200 to be exhausted through the Water Exhaust Port 52b.

In an aspect, while it is important for the inlet and exhaust ports 52a, 52b to remain unblocked, the ground and surface water monitoring system 10, and more specifically the WCS 100, can be configured to operate without causing damage to itself if either port 52a, 52b becomes blocked for an indefinite period of time. In an aspect, a particle filter can be placed in proximity to the intake port 52a. In another aspect, a pressure sensor can be used to measure the pressure occurring along the different water paths. If the pressure sensor finds that a path is experiencing pressure outside of allowable ranges, the sensor can be configured to shut off the pump 110 directly or through reporting the results to the system controller 300, which can be configured to control the operation of the pump 110. Further, the filter 104 does not have unlimited capacity and should be replaced as a part of normal maintenance.

FIGS. 5-8 illustrate components of the chemical sensor module (CSM) 200. The CSM 200 utilizes optical interferometry to sense the amount and makeup of contaminants in water that is passed through it. While the preferred embodiment of the present invention utilizes a Mach-Zehnder interferometer, other optical interferometers, including, but not limited to, Michelson, Fabry-Perot, Twyman-Green, Sagnac, Rayleigh, and Jamin interferometers can be used. The interferometer includes sensing channels coated with a polymer that selectively changes its index of refraction when exposed to contaminants found in the water.

Water, labeled as “water flow” at the top of FIG. 5, is pumped in from the right through an inlet tube 210, and passes through a sealed chamber (i.e. flowcell) 220 over the waveguide 230. At the same time, a low power laser 240, which in one embodiment has a center wavelength of 670 nm, launches a beam of monochromatic light into the waveguide 230. Interference patterns are displayed on a camera 250 or otherwise captured by another form of optical detector, at the output side of the CSM. For example, one embodiment of the present invention utilizes a UI-1542LE-M model camera from IDS Imaging. Other embodiments of the present invention can utilize other cameras 250.

In an aspect, as illustrated in FIGS. 6-8, the waveguide 230 is a stacked thin film structure with a base of optical glass, a thin core layer of a higher-index material, and an upper cladding layer into which are etched the long, narrow channels 234, 236. The waveguide 230, shown from a top view in FIG. 6, consists of an input grating 232, one or more pairs of parallel channels—sense 234 and reference 236—and one or more output gratings 238. In an aspect, the waveguide 230 can be comprised of, but is not limited to, fused silica glass, quartz glass, silicon, and tantalum pentoxide. The CSM 200 can be configured for the laser 240 to be oriented under the waveguide 230 so that the light of the laser 240 enters the input grating 232 from the bottom of the waveguide 230, and is refracted to travel down the length of the sense channel 234 and reference channel 236 (left to right in FIGS. 5-8).

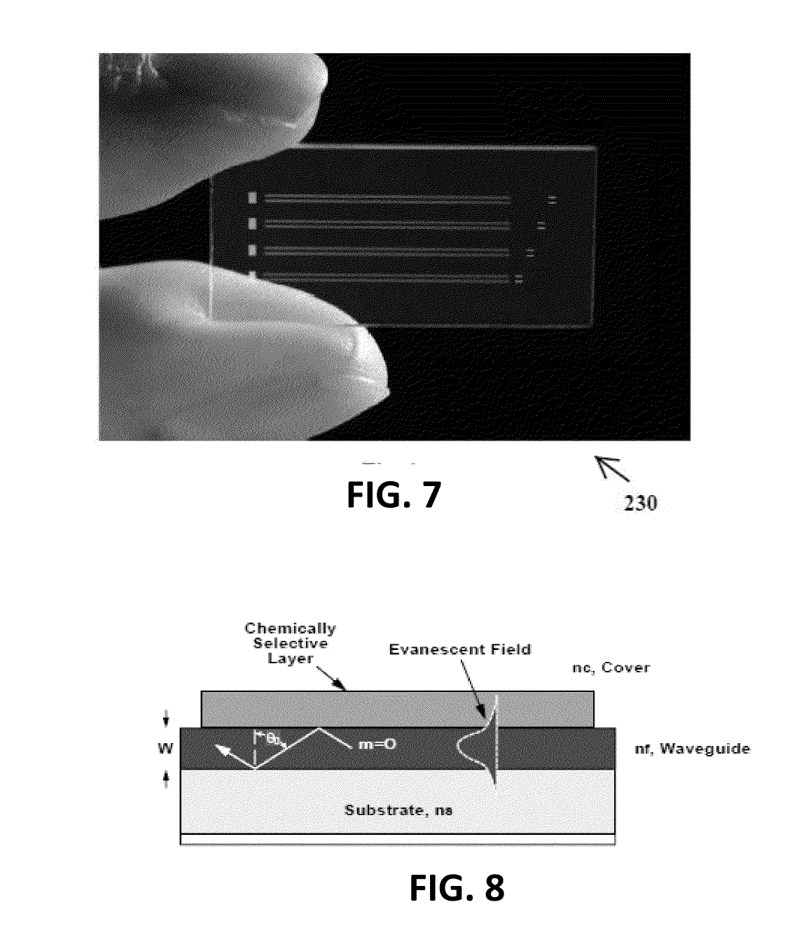

FIG. 7 is a top view photograph of a waveguide 230 having four pairs of sense 234 and reference 236 channels. The laser's light enters the input grating from the bottom of the waveguide 230, and is refracted to travel down the length of the channels (left to right in FIGS. 5 and 6).

In an aspect, the sense channel(s) 234 is/are coated with a chemically sensitive polymer, whose index of refraction changes in proportion to contaminant adsorption, causing the speed of light in that polymer to change correspondingly. A portion of the light (its evanescent field, as shown in FIG. 8) travels through the polymer coating that has been applied to the surface of the sense channel. A top coat can be used to cover the sense channel 234 to increase or decrease its affinity to certain chemical targets or interferents. Further, the chemically-sensitive polymer that is utilized is configured to adsorb the chemical as the polymer is exposed to the chemical, and desorbs as the polymer is exposed to a fluid without the chemical. The interference pattern generated by the optical interaction of light from these two channels is imaged on a CCD camera. The phase shift of this interference pattern, which is (ideally) proportional to the concentration of the target contaminant, is determined using a processor running image processing algorithms.

For example, the polymers that can be used to identify the type(s), concentration, and composition of contaminants in the water include, but are not limited to, PVP, PVA, PHEM, PVB, PTBrS, PVF, PEI, PVPy, PHPC, PBIBMA, TAF, PDMA, PMOS, PSSA, PVPK, and PIB. The types of contaminants that can be identified include, but are not limited to, benzene, toluene, other VOCs including those from the chlorinated ethylene family (e.g., TCE, PCE, vinyl chloride, cis- and trans-DCE), xylene, ammonia, hexane, chloramine, acetone, methylene chloride, chlorine, methanol, chloroform, hypochlorous acid, HCl, Freon, methane, ethane, ethylene, acetylene, nicotine, nitrates, phosphates, DMAC, DMMP, methyl salicylate, and the like. Further, the thickness of the polymer can vary as well, generally from less than a thousand to several thousand angstroms, depending on the response time, reverse time, and interferent response or rejection desired.

In other embodiments of the invention, other coatings and thicknesses can be used. The speed of light in each channel 234, 236 will be different to a degree proportional to the amount of contaminants in the air sample. In the preferred embodiment, both channels 234, 236 are also covered with a protective coating that is permeable to the contaminants. The protective coating can include, but is not limited to, polytetrafluroethlylene.

In an aspect, as the sense channel(s) 234 and reference channel 236 are exposed to the contaminated water (i.e., the fluid that has gone through the unfiltered path 106, such path potentially containing a “neutral” filter which does not affect the chemical composition of the fluid but does balance pressure and flow through the unfiltered path with that of the filtered path 102), contaminants are adsorbed in the sense channel 234 in proportion to the amount of the exposure (i.e., the more time exposed to contaminant, the more contaminant is adsorbed). Once the waveguide 230 has been exposed for the desired time, the waveguide 230 can then be exposed to the filtered fluid (i.e., the fluid that has gone through the filtered path 102 and filter 104). Light from the laser 240 is coupled into the waveguide 230 and a portion of this coupled light (its evanescent field, as shown in FIG. 8) travels through the polymer coating that has been applied to the surface of the sense channel 234. Laser light also travels down the adjacent reference channel 236. The interference pattern generated by the optical interaction of light from the sense channel 234 and the reference channel 236 is imaged on the camera 250. The sense channel 234, and more specifically the contaminant sensitive polymer, will capture or release molecules of the contaminant in proportion to the concentration of contaminant in the fluid, changing the index of refraction of the contaminant sensitive polymer on the sense channel 234, which therefore alters, in a quantifiable way, the interference pattern created by the recombination of the light passing through the sense channel 234 and the reference channel 236. The phase shift of the interference pattern, which, in an exemplary aspect, is proportional to the concentration of the contaminant, is determined using a processor running image processing algorithms, discussed in more detail below.

After exiting the right-hand side of each channel 234, 236, both light beams are again refracted and combined by the output grating, projecting an interference pattern onto the surface of the two-dimensional camera 250 or other form of optical detector. If contaminants are present, the sense and reference light waves will travel at different speeds, and one will arrive at the output grating 238 before the other, causing a phase shift in the interference pattern on the camera chip 250 that is proportional to contaminant concentration. The EC 300 analyzes the image from the camera chip 250 and measures the phase shift (the movement in the interference pattern over time as the contaminant concentration changes) to determine the concentration and identification of contaminants using calibration coefficients associated with the sensor 20. These coefficients may be updated through various means as well.

While FIG. 6 depicts a single pair of channels on the waveguide, waveguides may also contain multiple channel pairs (as shown in FIG. 7), each with the same or different sense polymers, to sense multiple analytes in the water sample, or to increase specificity and reliability. The ratio and timing of the adsorption responses of a given contaminant in different polymers is often highly unique, and can be used to determine the identity of a contaminant, or if the composition of a contaminant mixture has changed. The addition of even more sense channels 234 with different polymers, and thus more polymer pairs, to a waveguide 230 allows for more ratios to be measured and an increasing number of contaminants in a complex mixture to be identified and quantified. A signature of response ratios across the collection of polymer pairs on the waveguide 230 can be established for any contaminant mixture.

In an aspect, the EC 300 is resident in the sensor housing 30 and is not accessible to a user except functionally via the user interface 40 or external connectors 50. The EC 300 includes the power control system of the sensor 20. It is preferred that the power control system include a current monitor to detect off-nominal conditions, discussed in more detail below. In an aspect, the EC 300 includes on-board memory. In an exemplary aspect, the memory of the EC 300 is configured to be of a nonvolatile type and provide enough on-board memory to store an extended history of readings, consistent with application requirements, at the shortest reading interval, which can be set by the user. In another aspect, the memory is erased on a first-in, first-out basis when the memory becomes full. In an aspect, the memory of the EC 300 is configured to include user-defined identification data and to maintain a system log file. In an exemplary aspect, the memory of the MC 300 is configured to have 16 k of user-defined identification data and at least 512 k to maintain a system log file. In an aspect, the EC 300 includes a real time clock (RTC) which continues to track time even when the system is powered down. It is preferred that the RTC shall maintain an accuracy of better than ±6 hours per year for up to three years.

The EC 300 is configured to carry out the following functions: control the WCS 100; control the CSM 200; process images received from the CSM 200, including image cropping; determine an appropriate measurement zone; determine the interference pattern period within that zone; interpret interference pattern data and correlate with calibration data to obtain an analyte concentration reading; control the Touchscreen Display 30; interface with the CTRL and auxiliary outputs 54, 58; interface with the user via the Touchscreen 40 to set options and conduct maintenance; manage power input to the system 10; detect system faults and respond to them; save contaminant concentration data to a time-stamped data file; save significant events to a System Log; and detect and react to exceptions and errors. These functions can be implemented and executed using various coding languages or through various application layer software, including, but not limited to, Labview software from National Instruments. The EC 300 function may be distributed across multiple processors or controller integrated circuits, located on one or more printed circuit boards.

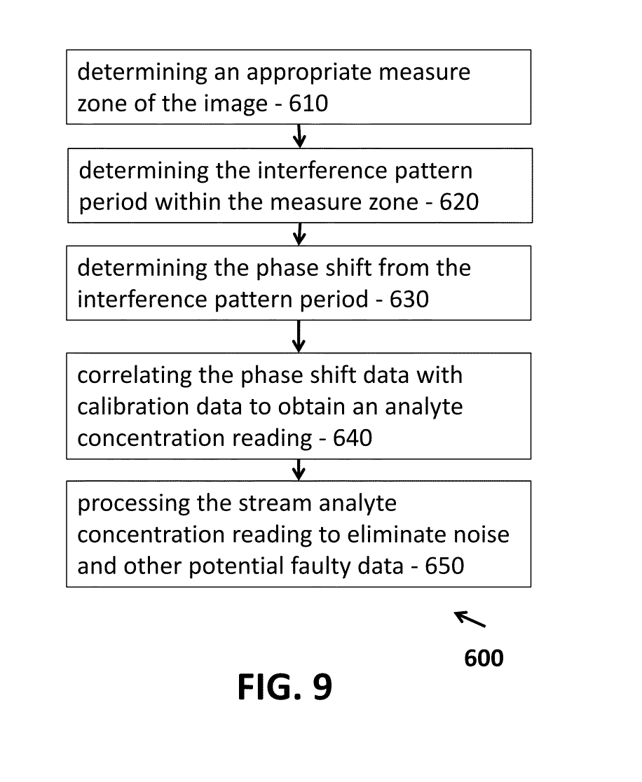

In an aspect, the EC 300 is further configured to control the operation of the WCS 100 and the CSM 200. In an aspect, the EC 300 is configured to control various functions of the CSM 200. In an aspect, the EC 300 can be configured to determine the contaminant concentration(s) as well. The EC 300 is configured to use applications, including a contaminant detection application discussed in detail below, to determine the contaminant(s) concentration. In addition, the EC 300 can be configured to process the images from the CSM 200, including image cropping, to determine the contaminant concentration. In an aspect, the EC 300 can determine the contaminant(s) concentration through a method 600 as illustrated in FIG. 9. In an aspect, the contaminant(s) concentration is done by determining an appropriate measurement zone of the image (step 610), determining the interference pattern period within the measurement zone (step 620), determining the phase shift that has occurred from the interference pattern period (step 630), correlating the phase shift data with calibration data to obtain an analyte concentration reading (step 640), and processing the stream analyte concentration reading to eliminate noise and other potential faulty data (step 650). In an exemplary aspect, the contaminant(s) detection application can perform the method 600.

The EC 300 can determine the appropriate measurement zone for the images collected by the camera 250 in various ways (step 610). In an aspect, the EC 300 can determine the appropriate measurement zone by evaluating the relative high and low intensities of the images captured by the camera 250. Other known methods can be used to determine the appropriate measure zone.

Once the measurement zone has been determined, the EC 300 can determine the interference pattern period within the measurement zone (step 620). In an aspect, the EC 300 can utilize image processing algorithms to determine the interference pattern period. In an exemplary aspect, the EC 300 can utilize a spatial Fourier transform algorithm. In such an aspect, the spatial Fourier transform algorithm is used to get the spatial frequency components of the interference pattern, and more specifically to find the dominant spatial frequency component. In other aspects, other algorithms or methods can be used to determine the interference pattern frequency and components other than the dominant frequency component of the interference pattern can be used to determine concentration.

Once the interference pattern's dominant spatial frequency has been determined (step 620), the EC 300 can find the phase shift that has occurred from the interference pattern period (step 630). In an exemplary aspect, the EC 300 can use the dominant spatial frequency component that was determined by the Fourier transform algorithm. In such an aspect, a phase demodulation can use the dominant frequency component to measure the phase shift. In other aspects, other processes can be used to determine the phase shift measurement.

Upon determining the phase shift measurement data, the EC 300 can then correlate the phase shift data with calibration data to obtain an analyte concentration reading (step 640). In an exemplary aspect, the phase shift data can be multiplied by a calibration coefficient to determine the ammonia concentration reading.

Once the analyte concentration reading is determined, the EC 300 can process the concentration reading to eliminate noise and other potential faulty data (step 650). This can be done by using weighted averaging algorithms or other signal processing techniques. In addition, other environmental conditions can be considered as well to eliminate faulty data. For example, the analyte concentration reading can be adjusted according to the current temperature or flow rate of the water. The analyte concentration reading can then be saved to the memory of the sensor 20 and/or displayed by the user interface 40.

AS discussed above, the EC 300 controls the operation of the monitoring system 10. The EC 300 is configured to provide simple operations for a user. As such, in the preferred embodiments of the system 10, the contaminant sensor 20 has a limited number of modes: a measurement mode, a standby mode, a system error mode, a maintenance mode, and a calibration and diagnostic mode. While it is preferred that the contaminant sensor 20 be limited to these five modes, other embodiments may include more optional modes, different modes, or fewer modes.

The screen illustrations in this section are provided to give an overview of each screen's contents. They are not meant to suggest specific layout or artwork for the screen. The screens shown are meant to correspond to the LCD Touchscreen area shown in FIG. 2. As shown in FIGS. 7 and 8, the display includes certain information provided to the user, including the status of which mode the contaminant sensor is presently operating, and any corresponding readings. For example, the display can include a measurement status indicator. As shown in FIG. 7, the measurement status indicator shows that the system is in Standby mode. In Standby mode, the contaminant sensor is not operational, and displays “STANDBY” on its screen.

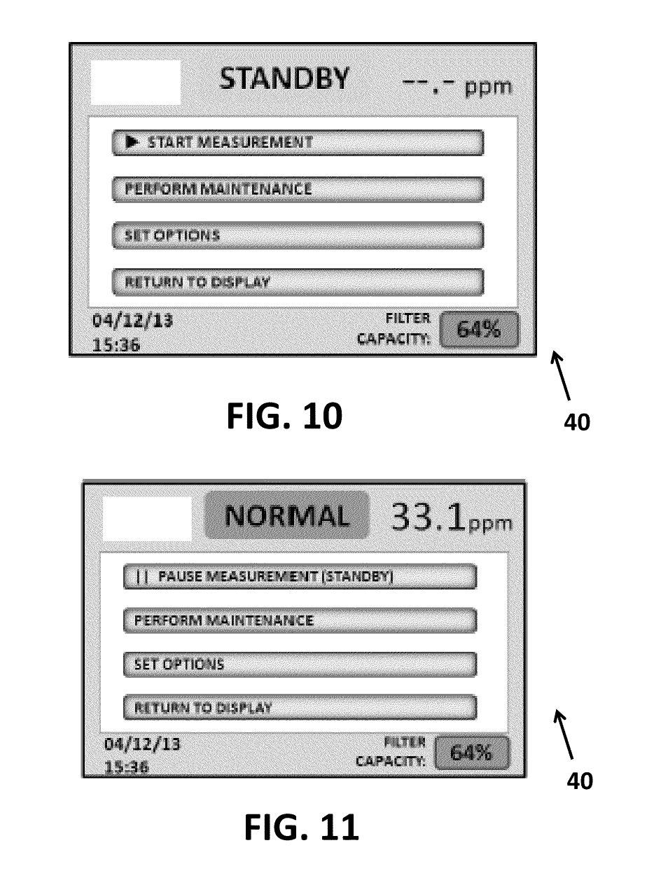

When in the measurement mode, the contaminant sensor continuously measures, displays, and records contaminant levels, presence, and compositions according to system presets. As shown in FIG. 10, the measurement status indicator shows that the system is in the MEASUREMENT mode, displaying a “NORMAL” with a green background that indicates the system is presently taking measurements, and the last measurement was within the Normal threshold set by the user. When the contaminant sensor finds that a last measurement of contaminant is above a Caution threshold set by the user, a “CAUTION” with a yellow background measurement status indicator is displayed. Likewise, when the contaminant sensor finds that a last measurement of contaminant is above a user-set Warning threshold, a “WARNING” with a red background measurement status indicator is displayed. Other embodiments may use descriptive words other than “Normal,” “Caution,” or “Warning.”

In addition to the measurement status indicator, the display includes a last measurement numerical indicator, a data/time display, and the filter capacity display. The numerical indicator indicates the last contaminant level reading taken. In Standby mode, this indicator reads “--.-”. Further, it is preferable that the numerical indicator display the amount with 0.1 ppm precision. The date/time display shows the present date and time, with minute precision, and is user adjustable in the preferred embodiment.

Also in the preferred embodiment, the date/time display adjusts for daylight savings time (US and Europe) and leap years. Lastly, the filter capacity display indicates the status, present capacity, or remaining life of the contaminant filter. In the preferred embodiment, the processor keeps track of the total amount of contaminants to which the filter has been exposed, as well as the time of exposure, and calculates remaining filter life. The numerical indicator is green for high capacity, transitions to yellow at a lower value, and then to red at a still lower value. When the filter capacity is at zero, the contaminant sensor will no longer take readings, and displays a “change filter” message. The default threshold values for the color changes can be changed based upon the user's preference. In the preferred embodiment, the number is automatically reset to its maximum each time the user goes through Maintenance mode. In some embodiments of the present invention, the filter capacity display will notify the user when a new filter has been installed improperly.

Referring to FIGS. 10-11, the display can be configured to include main menu controls. In an exemplary aspect, the main menu controls displayed can include a Start/Pause Measurement button, a Performance Maintenance button, a Set Options button, and a Return to Display button.

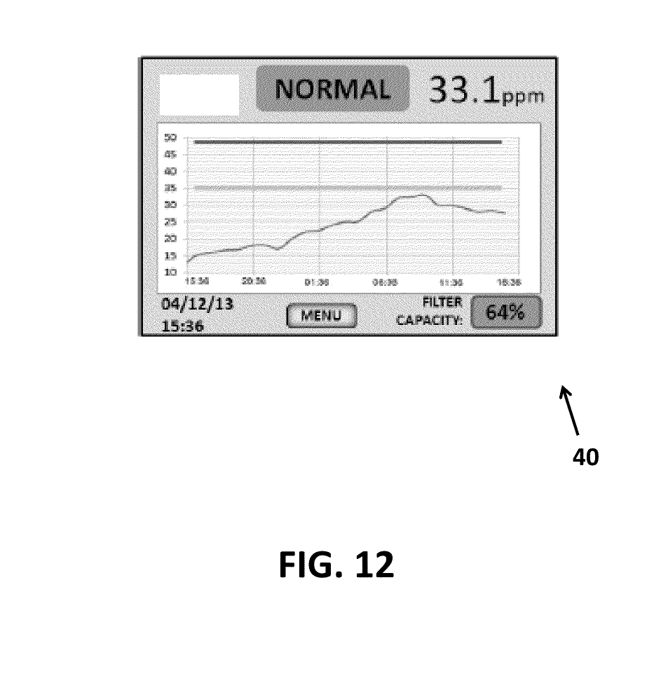

In an aspect, the Start/Pause Measurement button toggles between ‘Start’ and ‘Pause’ measurement. When pushed from Standby mode, it places the system in Measurement mode. When pushed from Measurement mode, it places the system in Standby mode. The Perform Maintenance button launches Maintenance mode, described in more detail below. The Set Options button opens a lower level of menus and keypad displays that allow the user to change system options. The Return to Display button returns to the graphical display of historical measured contaminant levels, as shown in FIG. 12. When in Standby Mode, the display is static and shows the most recent readings. When in Measurement Mode, the display is dynamic and continues to update. The Measurement Status Indicator, Last Measurement Numerical Indicator, Filter Capacity Indicator, and Date/Time Display continue to be displayed, independent of whether the graphical display or the menu is included in the embodiment.

As discussed above, the Graphical Display is initiated from the “Return to Display” button on the Main Menu. FIG. 12 illustrates the Graphical Display in Measurement Mode. As shown, the only control on the Graphical Display screen is the “Menu” button, which returns to the Main Menu (while remaining in either Standby or Measurement Mode). In other embodiments of the present invention, other buttons may be included on the Graphical Display.

The Graphical Display shows historical contaminant readings. In Measurement Mode, it updates in real time. In Standby Mode, it shows the most recent readings. The ‘x’ axis is time; ‘y’ axis is contaminant level in ppm (parts-per-million) or ppb (parts-per-billion). Both axes are user-adjustable and can auto-scale, as necessary and as desired, to accommodate the data. Yellow and red lines indicate the user-adjusted “Caution” and “Warning” thresholds, respectively. Date range limits on the display can be set by the user.

In an aspect, the parameters are displayed as a series of points connected by straight lines, during periods where the contaminant sensor 20 was in Measurement Mode. If the unit was placed in Standby Mode at any point during the time interval displayed, values during those durations are not displayed on the Display, appearing as gaps in the line. While FIG. 12 shows the display illustrating the numbers using a traditional x and y axis that correspond to time and contaminant levels, other types of graphical representations may be used in other embodiments, including, but not limited to, bar charts with min, max, and current levels, or pie charts.

In an aspect, the system 10 enters Measurement mode: (a) when “Start Measurement” is selected from the Main Menu; or (b) when the system 10 returns from System Error Mode, if Measurement Mode was the last known mode.

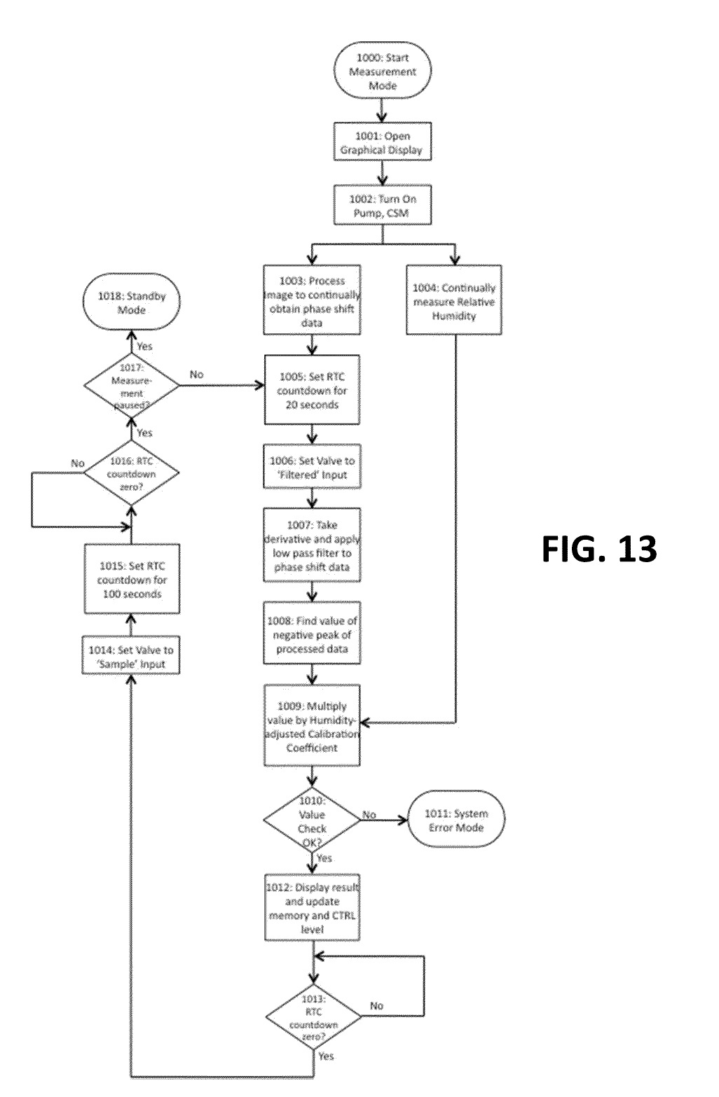

In Measurement Mode, the display 40 illustrates the Graphical Display as in FIG. 12. As always, the user may toggle between the Graphical Display and the Main Menu without leaving Measurement Mode. The contaminant sensor continuously takes contaminant level readings at the time interval specified by the Measurement Interval parameter, according to the behavioral flow shown in FIG. 13.

When the Measurement Mode is started (1000), the graphical display is changed (1001) to show something similar to that presented in FIG. 12. The pump, laser, and camera are turned on and initialized as appropriate (1002). Using image processing algorithms, which in one embodiment include the use of Fourier transforms, the system begins tracking the phase shift that occurs in the interference pattern detected by the camera (1003). Using the real-time clock, a counter is set (1005) for a duration (20 seconds for the preferred embodiment, but the duration can vary depending on the environment in which the ground and surface water monitoring system is utilized), and the valve is switched (1006) so that fluid having been filtered by the contaminant filter travels through the flow cell and over the waveguide. Phase shift data obtained by the image processing algorithms is further processed. According to one embodiment, the ground and surface water monitoring system first takes the derivative (or slope) of the data, and then “low-pass” filters the derivative data (1007). The maximum negative value of this processed data is then found (1008). Since the speed of the chemical adsorption and desorption of the contaminant responsive polymer coating is proportional to the presence and concentration of a specific contaminant in the sample, the derivative approach can be used. This approach preserves filter capacity. The value found in (1008) is then multiplied by a calibration coefficient (1009). The calibration coefficient can be provided at the time of installation of the waveguide in the sensor, and can be updated when needed at field calibration events. If the value obtained is within a reasonable range (which can be application dependent and defined at installation or by customers defining the out-of-bound reading levels) and not dramatically different from the last measurement (stored locally within the sensor) (1010), and if no other errors or failures are detected in the sensor, the value is then logged and passed along, or stored (1012). If the value is found not to be within the appropriate range, or an error is reported, the sensor enters System Error Mode (1011). Then, the system waits until the countdown has reached zero (1013) and, when this has occurred, the valve is switched so that the fluid is passed unfiltered through the flow cell to the waveguide (1014). The countdown clock is then reset (in the preferred embodiment, the clock is reset to 100 seconds) (1015), allowing the waveguide to adsorb and/or react to contaminant(s) from the water source. When the countdown again reaches zero (1016) and, assuming that the sensor has not been asked to pause or standby (1017), the sensor then resets the countdown and switches back to the filtered state (1005 and 1006). The system exits Measurement mode when: (a) the user selects “Pause Measurement Mode” from the Main menu; (b) a System Error occurs, or (c) the system is powered down.

The contaminant sensor enters Standby Mode when: (a) the system is powered up; (b) the user selects “Pause Measurement” from the Main Menu, or; (c) the system recovers from a System Error and the last state before the error was Standby Mode. FIG. 10 illustrates the Touchscreen in Standby Mode, when the Main Menu is being displayed. The Status Indicator is yellow, and reads “STANDBY”. The Numerical Indicator reads “--.- ppm”. The user may toggle between the Main Menu and the Graphical Display by hitting the “Return to Display” or “Menu” buttons. When the Graphical Display is shown during Standby mode, only historical data is presented, with the time intervals during which the system is in standby having no data appearing as gaps in the curve. In one embodiment of the present invention, if the user invokes Standby mode while the contaminant sensor is taking a measurement, the contaminant sensor interrupts the measurement, and then enters Standby Mode.

In Standby mode, the system 10 is configured as follows: Valve set to “Sense” (unfiltered) input, to minimize flow through the contaminant filter; Pump off; CSM laser and camera chip off; the CTRL output holds a ‘no data’ reading.

The contaminant sensor 20 enters System Error mode when it encounters certain error conditions. In some embodiments of the present invention, the System Error Mode is identical to Standby Mode, except that it is initiated by certain System Errors that require measurements to stop in order to prevent possible damage to the contaminant sensor or the reporting of ‘junk’ data. In System Error Mode, the Status Indicator flashes red and reads “SYSTEM ERROR”.

The contaminant sensor 20 generally exits System Error Mode, and returns to the last saved mode, when the error condition is corrected, either automatically or by user action. The System Log records time and date for entry into, and exit from, System Error mode.

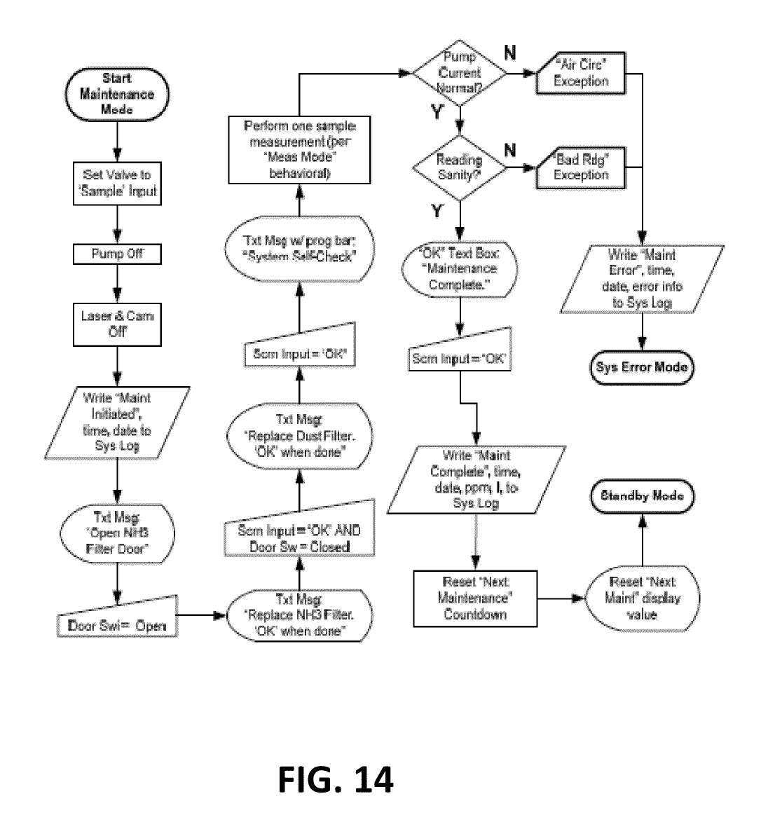

Maintenance mode places the system 10 in a safe state and guides the user through maintenance actions. Maintenance on the ground and surface water monitoring system 10 is intended to be carried out on a prescribed regular basis, but can be performed any time the user desires. FIG. 14 illustrates system behavior in Maintenance Mode. The User enters Maintenance mode by selecting “Perform Maintenance” from the main menu. The contaminant sensor configures the system as if in Standby Mode. A series of instructions on the Touchscreen guides the user through the maintenance steps, and the contaminant sensor performs several self-tests to ensure maintenance was carried out properly. The screen messages may include graphics that illustrate the task to be performed. The contaminant System Log records the start and end time/date of each maintenance.

The ground and surface water monitoring system may incorporate a maintenance countdown, which alerts the user to when the system needs regularly scheduled maintenance. In addition to keeping a ‘maintenance countdown’, the ground and surface water monitoring system may keep a separate ‘Replacement Countdown’, and shall alert the user via a Touchscreen text message when a replacement contaminant sensor is due.

Calibration and Diagnostic mode facilitates calibrating the unit and performing certain diagnostics. The user initiates Calibration and Diagnostic mode by connecting a computer to the USB port and running a Calibration & Diagnostic application from the computer. The application calibrates the chemical sensor by exposing the contaminant sensor to a test fixture containing known levels of contaminants mixed with the water/fluid, as verified by a reliable, high-precision reference sensor. The software application may be configured to guide the operator through the calibration steps, and generate appropriate calibration coefficients, based on formulas and/or lookup tables created during product development. Calibration coefficients are stored locally on the contaminant sensor, as well as in a global database that references calibration coefficients to device serial number and date. Calibration coefficients are preferred to remain valid for at least three years, with no updates.

The calibration application will also load identifying data into the contaminant sensor's local memory, as well as the global database. Identifying data includes device hardware revision, firmware revision, serial number, date of manufacture, CSM serial number, and Waveguide Serial Number, with space reserved for user-defined data. Other identifying data may be included as well.

The “Cal & Diagnostic” application also has the ability to retrieve, store, and display real-time diagnostic data from the contaminant sensor, to assist in troubleshooting and understanding system behavior. Parameters may include, but are not limited to, pump current draw, total current draw, CSM current draw, CRC scan results, measurement history, system log, raw image data, and manual control of various subsystems.

Selecting “Set Options” from the Main Menu opens a new menu with a number of options available to the user. These include “Caution” and/or “Warning” thresholds whose levels, within certain application-specific ranges, can be adjusted by the user.

As discussed above, the ground and surface water monitoring system may include a USB memory stick containing a simple application to be installed on a computer for downloading and displaying data from the contaminant sensor. An installation program may guide the user through the process. To download data from the contaminant sensor, the user connects the memory stick to the sensor's USB port, waits for a “data download complete” message on the touchscreen, then removes the memory stick and connects it to the computer. Connection and data download can take place in any contaminant sensor mode, without interrupting the contaminant sensor measurements or other functions. A simple interface will allow the user to: view date ranges available for download; select a date range and download the data for import to a program such as Microsoft Excel or other spreadsheet applications; display the data graphically for a selected date range; and/or download a copy of the System Log.

If the computer is connected to the Internet, the application will, with user's permission, connect to a designated website and check for available firmware upgrades. If one is available, the application will download and install it to the memory stick. When the memory stick is inserted into the contaminant sensor again, it will upgrade the system software. If the contaminant sensor is in Measurement mode, the application will complete the present measurement and place the system in Standby mode during upgrade, then automatically return the system to Measurement mode. Sensor software may also be upgraded directly if the sensor is connected to the Internet.

The EC 300 of the contaminant sensor maintains a System Log for historical and diagnostic purposes according to one embodiment of the present invention. The System log shall be available for download, and is viewable from the Options screen. It shall contain time-stamped records of all significant system events. The timestamp shall be independent of the user's clock setting. Examples of ‘significant system events’ are: Maintenance start/stop times; System Errors; Start/Stop Measurement Mode; User Data Downloads; Options Changes by User; and Power-ups.

The control firmware of the EC 300 of the contaminant sensor is configured to handle exceptions and off-normal events during its operation. These events will not cause system instability, hardware damage, or an unsafe situation. Exceptions are normally handled by error messaging prompting the user to take action.

In other embodiments of the present invention, the location and association of the components of the ground and surface water monitoring system may vary from what is described above. For example, in one embodiment of the present invention, the various pumps, valves, and hoses can be exposed, or be contained within the housing of the sensor.

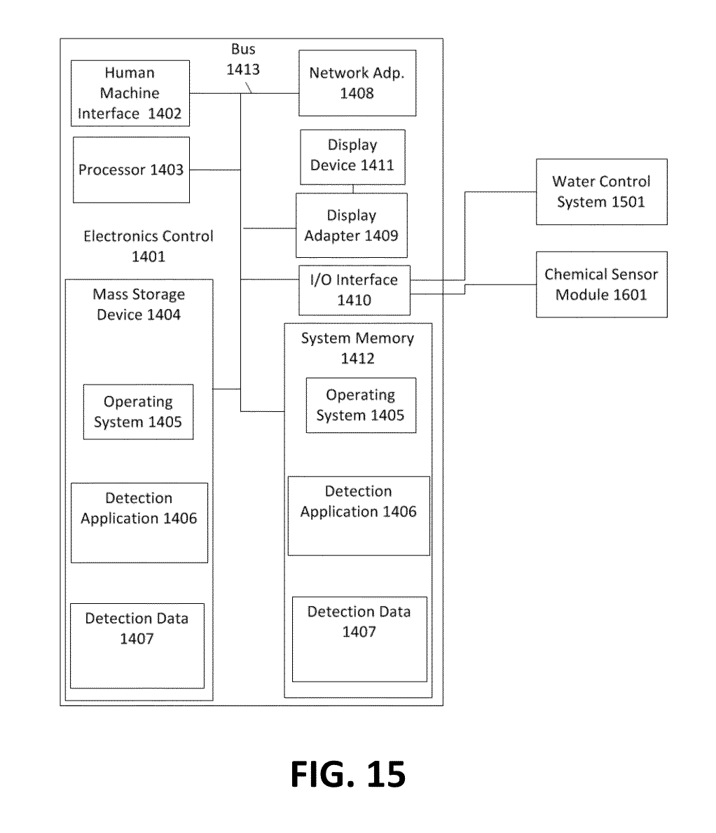

FIG. 15 is a block diagram illustrating an exemplary operating environment for performing the disclosed methods. This exemplary operating environment is only an example of an operating environment and is not intended to suggest any limitation as to the scope of use or functionality of operating environment architecture. Neither should the operating environment be interpreted as having any dependency or requirement relating to any one or combination of components illustrated in the exemplary operating environment.

The present methods and systems can be operational with numerous other general purpose or special purpose computing system environments or configurations. Examples of well-known computing systems, environments, and/or configurations that can be suitable for use with the systems and methods comprise, but are not limited to, personal computers, server computers, laptop devices, and multiprocessor systems. Additional examples comprise set top boxes, programmable consumer electronics, network PCs, minicomputers, mainframe computers, distributed computing environments that comprise any of the above systems or devices, and the like.

The processing of the disclosed methods and systems can be performed by software components. The disclosed systems and methods can be described in the general context of computer-executable instructions, such as program modules, being executed by one or more computers or other devices. Generally, program modules comprise computer code, routines, programs, objects, components, data structures, etc., that perform particular tasks or implement particular abstract data types. The disclosed methods can also be practiced in grid-based and distributed computing environments where tasks are performed by remote processing devices that are linked through a communications network. In a distributed computing environment, program modules can be located in both local and remote computer storage media including memory storage devices.

Further, one skilled in the art will appreciate that the systems and methods disclosed herein can be implemented via the electronic controller in the form of a computer 1401 as illustrated in FIG. 13 (the MC 1401 can be thought of as a general-purpose computing device like a computer board 1401 but contained within the sensor 20). The components of the EC 1401 can comprise, but are not limited to, one or more processors or processing units 1403, a system memory 1412, and a system bus 1413 that couples various system components including the processor 1403 to the system memory 1412. In the case of multiple processing units 1403, the system can utilize parallel computing.

The system bus 1413 represents one or more of several possible types of bus structures, including a memory bus or memory controller, a peripheral bus, an accelerated graphics port, and a processor or local bus using any of a variety of bus architectures. By way of example, such architectures can comprise an Industry Standard Architecture (ISA) bus, a Micro Channel Architecture (MCA) bus, an Enhanced ISA (EISA) bus, a Video Electronics Standards Association (VESA) local bus, an Accelerated Graphics Port (AGP) bus, and a Peripheral Component Interconnects (PCI), a PCI-Express bus, a Personal Computer Memory Card Industry Association (PCMCIA), Universal Serial Bus (USB) and the like. The bus 1413, and all buses specified in this description can also be implemented over a wired or wireless network connection and each of the subsystems, including the processor 1403, a mass storage device 1404, an operating system 1405, detection application 1406, detection data 1407 (including the contaminant concentration amounts, thresholds, etc.), a network adapter 1408, system memory 1412, an Input/Output Interface 1410, a display adapter 1409, a display device 1411, and a human machine interface 1402.

The EC 1401 can comprise a variety of computer readable media. Exemplary readable media can be any available media that is accessible by the EC 1401 and comprises, for example and not meant to be limiting, both volatile and non-volatile media, removable and non-removable media. The system memory 1412 comprises computer readable media in the form of volatile memory, such as random access memory (RAM), and/or non-volatile memory, such as read only memory (ROM). The system memory 1412 typically contains data such as detection data 1407 and/or program modules such as operating system 1405 and detection application 1406 that are immediately accessible to and/or are presently operated on by the processing unit 1403.

In another aspect, the EC 1401 can also comprise other removable/non-removable, volatile/non-volatile computer storage media. By way of example, FIG. 14 illustrates a mass storage device 1404, which can provide non-volatile storage of computer code, computer readable instructions, data structures, program modules, and other data for the computer 1401. For example and not meant to be limiting, a mass storage device 1404 can be a hard disk, a removable magnetic disk, a removable optical disk, magnetic cassettes or other magnetic storage devices, flash memory cards, CD-ROM, digital versatile disks (DVD) or other optical storage, random access memories (RAM), read only memories (ROM), electrically erasable programmable read-only memory (EEPROM), and the like.

Optionally, any number of program modules can be stored on the mass storage device 1404, including by way of example, an operating system 1405 and detection application 1406. Each of the operating system 1405 and detection application 1406 (or some combination thereof) can comprise elements of the programming and the detection application 1406. Detection data 1407 can also be stored on the mass storage device 1404. Detection data 1407 can be stored in any of one or more databases known in the art. Examples of such databases include DB2®, Microsoft® Access, Microsoft® SQL Server, Oracle®, mySQL, PostgreSQL, and the like. The databases can be centralized or distributed across multiple systems.

In another aspect, the user can enter commands and information into the EC 1401 via an input device (not shown). Examples of such input devices comprise, but are not limited to, a keyboard, pointing device (e.g., a “mouse”), a microphone, a joystick, a scanner, tactile input devices such as gloves, and other body coverings, and the like. These and other input devices can be connected to the processing unit 1403 via a human machine interface 1402 that is coupled to the system bus 1413, but can be connected by other interface and bus structures, such as a parallel port, game port, an IEEE 1394 Port (also known as a Firewire port), a serial port, or a universal serial bus (USB).

In yet another aspect, a display device 1411 can also be connected to the system bus 1413 via an interface, such as a display adapter 1409. In an aspect, the display device 1411 can be the interface 40 shown in FIG. 2. It is contemplated that the EC 1401 can have more than one display adapter 1409 and the EC 1401 can have more than one display device 1411. For example, a display device can be a monitor, an LCD (Liquid Crystal Display), or a projector. In addition to the display device 1411, other output peripheral devices can comprise components such as speakers (not shown) and a printer (not shown) which can be connected to the computer 1401 via Input/Output Interface 1410. Any step and/or result of the methods can be output in any form to an output device. Such output can be any form of visual representation, including, but not limited to, textual, graphical, animation, audio, tactile, and the like.

As discussed above, the EC 1401 can operate and control a water control system (WCS) 1501 and a chemical sensor module (CSM) 1601. The EC 1401 can be connected to the WCS 1501 and CSM 1601 through various input/output interfaces 1410.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; the number or type of embodiments described in the specification.

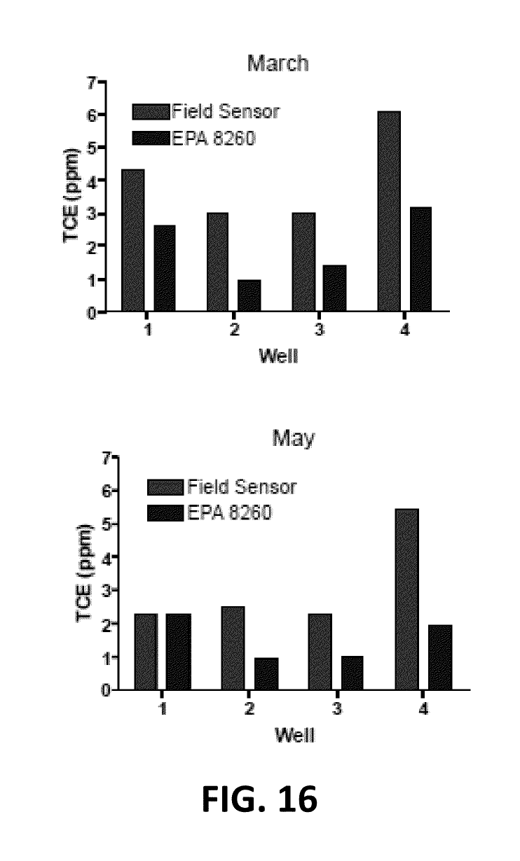

An embodiment of the present invention was tested with favorable results, discussed herein. A sensor 20 with a waveguide 230 having a single polymer sensing film was field-tested at a major industrial site having significant TCE contamination in the groundwater. Sensor tests were performed in parallel with both conventional (EPA Method 8260) sampling and analysis protocol, as well as with an onsite UV-VIS spectrometer. Triplicate samples of groundwater were pumped up from several monitor wells 12 and tested using the sensor 20. The sensor's phase shift readings were converted to TCE concentration based on laboratory calibration data. The measured concentrations, as well as data obtained from the parallel laboratory measurements using Method 8260, are shown in FIG. 16. While not shown, there was excellent agreement between results from the sensor and results from the UV-VIS spectrometer. FIG. 16 also shows the change recorded at these same four different wells over a two-month time interval. In all cases, the concentration measured using the sensor follows the trend of the analytical results but was consistently higher. This is attributable to the nearly unavoidable loss of TCE during standard groundwater well sampling techniques and the necessary transfers and sample manipulations involved in laboratory analysis.

The first benefit of a ground and surface water monitoring system as discussed above is a major reduction in the expense of monitoring groundwater sites. The installation of the ground and surface water monitoring system will eliminate the need for repeat trips by monitoring personnel, resulting in significant reduction in personnel time. For example, TCE annual monitoring cost has been reported to be over $600,000, with the average annual cost per monitoring well of $2,800, at the former Dobbins Air Force Base site near Atlanta, Ga. At such a cost, it is estimated that every one thousand monitoring wells or surface monitoring points with sensors deployed would result in annual savings of about $1.5 million.

A second benefit is the significant economic value of ‘continuous monitoring’ at contamination sites and at the inputs for drinking water systems close to contaminated sites. If treatment systems or monitoring sites experience contamination exceeding thresholds set by the user, the sensor will raise an immediate alarm, which could shut down the water system. For locations where the groundwater contamination poses a potential threat of vapor intrusion into buildings, continuous sensing at the perimeter of the plume would instigate immediate remediation activities to stabilize the plume.

Continuous onsite monitoring also eliminates the inaccuracies due to sample degradation upon collection, transport, and analysis. Sampling protocols quite often result in an underreporting of true contamination levels. Furthermore, onsite sensors offer researchers the opportunity to monitor microbial and chemical reactions in real-time, thus shortening test cycles and feedback loops, and allowing researchers to more quickly gain insights into contaminant behavior. Real-time monitoring of remediation scenarios will serve to expedite cleanup times and further reduce costs.

A third significant benefit can be the detection of a wide range of harmful contaminants to include the BTEX chemicals, nitrates and nitrites, and phosphates, and even microbial intruders. We expect that, at this point, the sensor would be widely deployed into water systems of all types, including industrial, municipal, and even private systems.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above described embodiment, method, and examples, but by all embodiments and methods within the scope and spirit of the invention. To the extent necessary to understand or complete the disclosure of the present invention, all publications, patents, and patent applications mentioned herein are expressly incorporated by reference therein to the same extent as though each were individually so incorporated.

Having thus described exemplary embodiments of the present invention, those skilled in the art will appreciate that the within disclosures are exemplary only and that various other alternatives, adaptations, and modifications may be made within the scope of the present invention. Accordingly, the present invention is not limited to the specific embodiments as illustrated herein, but is only limited by the following claims.

What is claimed is:

1. An automated water source monitoring system, comprising:
a. an interferometer configured to track the amount of a contaminant in a fluid; and
b. a device for supplying a representative flow of the fluid to the interferometer.
